(19) United States
(12) Patent Application Publication
(10) Pub. No.: US 2013/0190610 A1
(43) Pub. Date: Jul. 25, 2013

(54) ULTRASOUND DIAGNOSTIC APPARATUS AND METHOD

(71) Applicant: GE MEDICAL SYSTEMS GLOBAL TECHNOLOGY COMPANY, LLC, Waukesha, WI (US)

(72) Inventor: Lei Liu, Tokyo (JP)

(73) Assignee: GE MEDICAL SYSTEMS GLOBAL TECHNOLOGY COMPANY, LLC, Waukesha, WI (US)

(21) Appl. No.: 13/744,034

(22) Filed: Jan. 17, 2013

(30) Foreign Application Priority Data
Jan. 19, 2012 (JP) 2012-008748

(51) Int. Cl. A61B 8/08; A61B 8/00; A61B 8/14; A61B 10/02

(52) U.S. Cl. CPC A61B 8/0841; A61B 10/0233; A61B 8/483; A61B 8/4245; A61B 8/4444; A61B 8/488; A61B 8/463; A61B 8/14; A61B 8/466; A61B 8/52
USPC 600/424

(57) ABSTRACT

An ultrasound diagnostic apparatus is provided. The ultrasound diagnostic apparatus includes a fluid position detection unit configured to detect a position of fluid in a subject, a biopsy needle position detection unit configured to detect a position of a biopsy needle to be inserted into the subject, a distance calculation unit configured to calculate a distance between the fluid and the biopsy needle, based on the detected position of the fluid and the detected position of the biopsy needle, and a notice unit configured to generate a warning when the distance calculated by the distance calculation unit is smaller than a predetermined threshold value.

### CROSS REFERENCE TO RELATED APPLICATIONS

This application claims the benefit of Japanese Patent Application No. 2012-008748 filed Jan. 19, 2012, which is hereby incorporated by reference in its entirety.

### BACKGROUND OF THE INVENTION

The present invention relates to an ultrasound diagnostic apparatus which aids in the insertion of a biopsy needle by an operator, and a method thereof.

A biopsy needle may be inserted into a subject to perform sampling of a biological tissue and radiofrequency wave cautery treatment. It is necessary to prevent vasa such as blood vessels from being damaged upon the insertion of the biopsy needle. Since an ultrasound diagnostic apparatus is capable of displaying an ultrasound image of a subject in real time while performing the transmission/reception of ultrasound to and from the subject, the insertion of the biopsy needle is performed while confirming the ultrasound image (refer to, for example, Japanese Unexamined Patent Publication No. 2011-229837).

Expert skills are however required to insert the biopsy needle in such a manner as to avoid damage of vasa while confirming the ultrasound image. There has thus been a demand for an ultrasound diagnostic apparatus capable of making it easier for a person who performs a biopsy manipulation to insert the biopsy needle.

### BRIEF DESCRIPTION OF THE INVENTION

In one aspect, an ultrasound diagnostic apparatus is provided. The ultrasound diagnostic apparatus includes a fluid position detection unit which detects a position of fluid in a subject, a biopsy needle position detection unit which detects a position of a biopsy needle to be inserted into the subject, a distance calculation unit which calculates a distance between the fluid and the biopsy needle, based on the position of the fluid, which has been detected by the fluid position detection unit and the position of the biopsy needle, which has been detected by the biopsy needle position detection unit, and a notice unit which notifies a warning when the distance calculated by the distance calculation unit is smaller than a predetermined threshold value.

According to the above aspect, since the warning is notified where the distance calculated by the distance calculation unit is smaller than the predetermined threshold value, the insertion of the biopsy needle into the subject can be performed easier.

Further advantages will be apparent from the following description of the exemplary embodiments as illustrated in the accompanying drawings.

### BRIEF DESCRIPTION OF THE DRAWINGS

FIG. 1 is a block diagram showing one example of a schematic configuration of an embodiment of an ultrasound diagnostic apparatus.

FIG. 2 is a block diagram illustrating a configuration of an echo data processor in the ultrasound diagnostic apparatus shown in FIG. 1.

FIG. 3 is a block diagram depicting a configuration of a display controller in the ultrasound diagnostic apparatus shown in FIG. 1.

FIG. 4 is a diagram showing one example of a display unit on which a B-mode image is displayed.

FIG. 5 is a diagram illustrating one example of the display unit on which character information about a warning is displayed.

FIG. 6 is a diagram depicting one example of the display unit on which a 3D image is displayed.

FIG. 7 is a diagram showing one example of the display unit on which a 2D image is displayed.

FIG. 8 is a diagram showing another example of a 2D image.

FIG. 9 is a block diagram illustrating another example of a schematic configuration of an embodiment of an ultrasound diagnostic apparatus.

FIG. 10 is a diagram showing one example of a display unit on which a distance calculated by a distance calculation unit is displayed.

FIG. 11 is a diagram illustrating another example of the display unit on which a 3D image is displayed in a fourth modification.

FIG. 12 is a diagram depicting a further example of the display unit on which a 2D image is displayed in the fourth modification.

FIG. 13 is a block diagram showing a configuration of an echo data processor in a fifth modification.

FIG. 14 is a block diagram illustrating a configuration of a controller in a sixth modification.

### DETAILED DESCRIPTION OF THE INVENTION

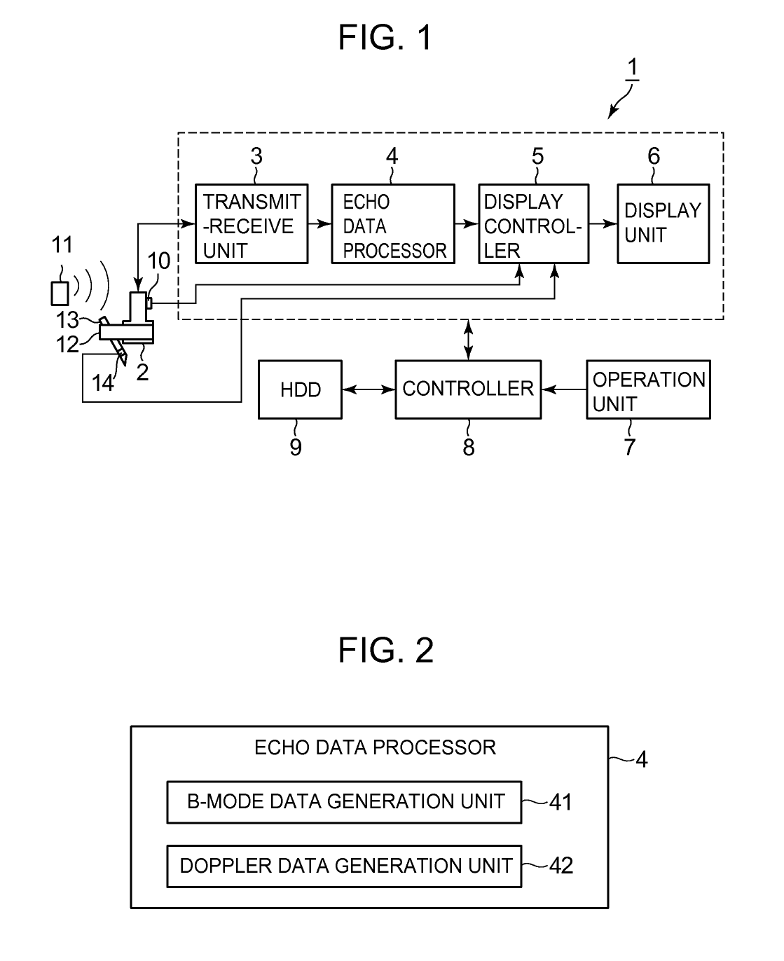

An exemplary embodiment will be described below based on FIGS. 1 through 6. An ultrasound diagnostic apparatus 1 shown in FIG. 1 is equipped with an ultrasound probe 2, a transmit-receive unit 3, an echo data processor 4, a display controller 5, a display unit 6, an operation unit 7, a controller 8 and an HDD (Hard Disk Drive) 9.

The ultrasound probe 2 includes a plurality of ultrasound transducers (not shown) arranged in array form. The ultrasound probe 2 transmits ultrasound to a subject through the ultrasound transducers and receives its echo signals.

The ultrasound probe 2 is provided with a first magnetic sensor 10 including, for example, a Hall element. The first magnetic sensor 10 detects magnetic field generated from a magnetic generation unit 11 that includes, for example, a magnetic generating coil. A signal detected by the first magnetic sensor 10 is inputted to the display controller 5. The signal detected by the first magnetic sensor 10 may be inputted to the display controller 5 through an unillustrated cable or may be inputted to the display controller 5 by radio. The magnetic generation unit 11 and the first magnetic sensor 10 are used to detect the position and tilt of the ultrasound probe 2 as will be described later.

A biopsy needle 13 is attached to the ultrasound probe 2 by means of a biopsy needle guide jig 12. The biopsy needle 13 is provided with a second magnetic sensor 14 which detects magnetic field generated from the magnetic generation unit 11. The second magnetic sensor 14 is provided in a hollow portion of a tip portion of the biopsy needle 13, which is shaped in cylindrical form, for example. The magnetic generation unit 11 and the second magnetic sensor 14 are used to detect the position of the tip portion of the biopsy needle 13 provided with the second magnetic sensor 14.

The transmit-receive unit 3 supplies an electric signal for transmitting ultrasound from the ultrasound probe 2 under a predetermined scan condition to the ultrasound probe 2, based on a control signal outputted from the controller 8. The transmit-receive unit 3 performs signal processing such as A/D conversion, phasing-adding processing, etc. on each echo signal received by the ultrasound probe 2 and outputs echo data after the signal processing to the echo data processor 4.

The echo data processor 4 performs processing for generating an ultrasound image on the echo data outputted from the transmit-receive unit 3. For example, the echo data processor 4 has a B-mode data generation unit 41 and a Doppler data generation unit 42 as shown in FIG. 2. The B-mode data generation unit 41 performs B-mode processing including logarithmic compression processing, envelop detection processing or the like on the echo data to thereby generate B-mode data.

The Doppler data generation unit 42 performs Doppler processing including quadrature detection processing, autocorrelation arithmetic processing or the like on the echo data to thereby generate Doppler data. This Doppler data is data about a flow rate and variance of an echo source, for example. Alternatively, the Doppler data may be data about the power of an echo source. The Doppler data is one example of an embodiment of fluid information data. The Doppler data generation unit 42 is one example of an embodiment of a fluid information data generation unit.

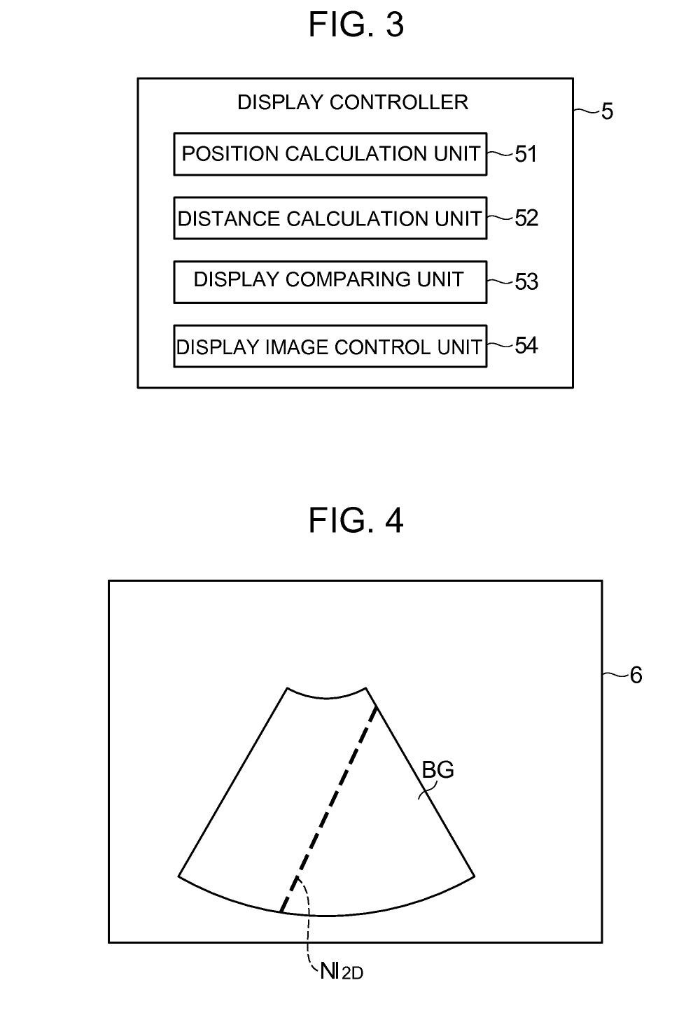

The display controller 5 has a position calculation unit 51, a distance calculation unit 52, a distance comparing unit 53 and a display image control unit 54 as shown in FIG. 3. The position calculation unit 51 calculates information (hereinafter called “probe position information”) about the position and tilt of the ultrasound probe 2 in a three-dimensional space with the magnetic generation unit 11 as an origin point, based on the magnetic detection signal from the first magnetic sensor 10. Further, the position calculation unit 51 calculates position information about each echo data in the three-dimensional space, based on the probe position information. Thus, position information about Doppler data in the three-dimensional space is calculated, and position information about fluid such as a blood flow is obtained.

The position of the fluid is detected by the first magnetic sensor 10, the magnetic generation unit 11, the Doppler data generation unit 42 and the position calculation unit 51 (fluid position detecting function).

The position calculation unit 51 calculates position information about the tip portion of the biopsy needle 13 in the three-dimensional space, based on the magnetic detection signal outputted from the second magnetic sensor 14. The position of the tip portion of the biopsy needle 13 is detected by the second magnetic sensor 14, the magnetic generation unit 11 and the position calculation unit 51 (biopsy needle position detecting function).

The three-dimensional space with the magnetic generation unit 11 as the origin point is one example of an embodiment of a three-dimensional space.

The distance calculation unit 52 calculates a distance between the fluid and the biopsy needle 13. The distance calculation unit 52 calculates a distance D between the tip portion of the biopsy needle 13 provided with the second magnetic sensor 14 and the fluid (distance calculating function). The distance calculation unit 52 calculates, as the distance D, a distance between the position of the Doppler data in the three-dimensional space and the position of the tip portion of the biopsy needle 13. The details thereof will be described later.

The distance comparing unit 53 compares the distance D calculated by the distance calculation unit 52 and predetermined threshold values. The predetermined threshold values are a first threshold value Dth1 and a second threshold value Dth2 (where Dth1<Dth2). Their details will be described later.

Incidentally, the first threshold value Dth1 and the second threshold value Dth2 have been stored in the HDD 9 in advance. The first threshold value Dth1 and the second threshold value Dth2 may be changed by the input of the operation unit 7 by an operator.

The display image control unit 54 scan-converts the B-mode data to B-mode image data by a Scan Converter. Then, the display image control unit 54 causes the display unit 6 to display a B-mode image based on the B-mode image data.

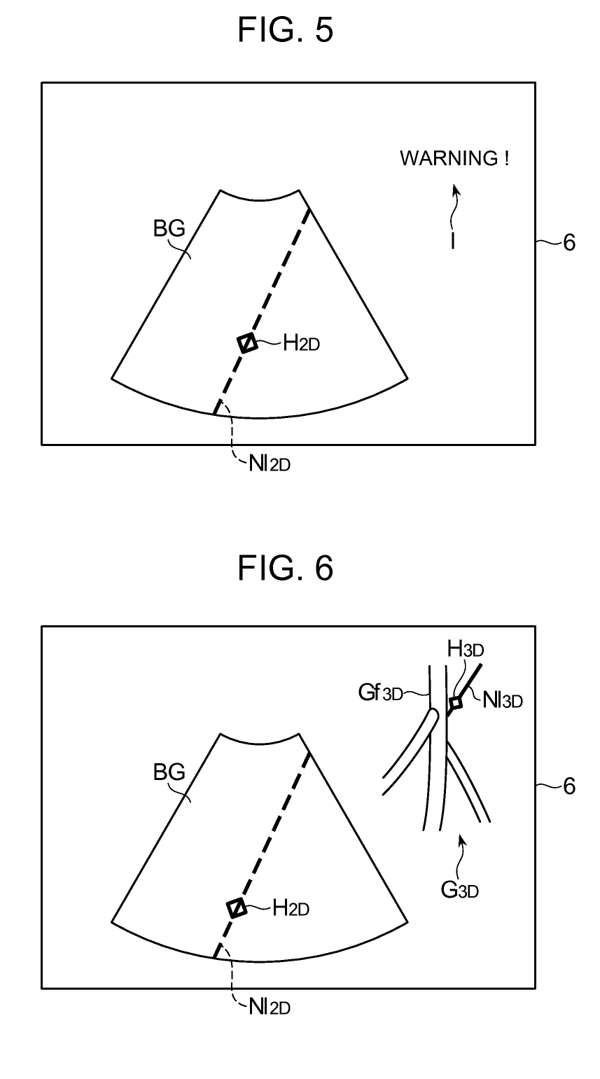

The display image control unit 54 causes the display unit 6 to display images of warning when the distance D reaches the predetermined threshold value or less as will be described later (warning displaying function). The warning images are character information I about a warning (refer to FIG. 5) and a 3D image G_{3D} (refer to FIG. 6).

The character information I about the warning is a message which warns that the biopsy needle 13 is approaching fluid, and is a character of “WARNING!”.

The 3D image G_{3D} includes a three-dimensional fluid image Gf_{3D}, a three-dimensional needle line N1_{3D} indicative of an insertion estimated path of the biopsy needle, and a three-dimensional tip display H_{3D} indicative of the tip portion of the biopsy needle 13. The display image control unit 54 generates three-dimensional fluid image data, based on the Doppler data and causes the display unit 6 to display the three-dimensional fluid image Gf_{3D} based on the three-dimensional fluid image data.

The display image control unit 54 specifies a positional relationship between the fluid in the three-dimensional space and the tip portion of the biopsy needle 13, based on the position information about the Doppler data and the position information about the tip portion of the biopsy needle 13, both of which have been calculated by the position calculation unit 51 and thereby causes the three-dimensional fluid image Gf_{3D} and the three-dimensional tip display H_{3D} to be displayed.

A positional relationship between the insertion estimated path of the biopsy needle 13 and the ultrasound probe 2 has been set in advance. Thus, the position of the insertion estimated path of the biopsy needle 13 in the three-dimensional space is specified based on the probe position information calculated by the position calculation unit 51. The display image control unit 54 specifies a positional relationship between the insertion estimated path of the biopsy needle 13, the fluid and the tip portion of the biopsy needle 13 and causes the three-dimensional needle line N1_{3D} to be displayed.

The display image control unit 54 causes a two-dimensional needle line N1_{2D} to be displayed on a B-mode image BG displayed on the display unit 6 as will be described later. The display image control unit 54 causes a two-dimensional tip display H_{2D} indicative of the tip portion of the biopsy needle 13 to be displayed on the B-mode image BG, based on the position information about the tip portion of the biopsy needle 13, which is calculated by the position calculation unit 51.

The display unit 6 includes an LCD (Liquid Crystal Display), a CRT (Cathode Ray Tube) or the like. The operation unit 7 includes a keyboard and a pointing device (not shown) or the like for inputting instructions and information by an operator.

The controller 8 has a CPU (Central Processing Unit). The controller 8 reads a control program stored in the HDD 9 to execute functions at the respective parts of the ultrasound diagnostic apparatus 1 starting with the fluid position detecting function, the biopsy needle position detecting function, the distance calculating function and the warning displaying function.

The operation of the ultrasound diagnostic apparatus 1 according to the exemplary embodiment will now be explained. The ultrasound diagnostic apparatus 1 first starts the transmission/reception of ultrasound through the ultrasound probe 2 to obtain each echo signal. The transmission/reception of ultrasound by the ultrasound probe 2 is performed on a three-dimensional region. Thus, the echo signal in the three-dimensional region is acquired.

A B-mode image BG generated based on the echo signal is displayed on the display unit 6 as shown in FIG. 4. The B-mode image BG is a two-dimensional image. The two-dimensional needle line N1_{2D} is displayed in the B-mode image BG.

The Doppler generation unit 42 generates Doppler data, based on the echo signal. The Doppler data generated here is three-dimensional data (volume data).

The operator inserts the biopsy needle 13 into a subject while seeing the real-time B-mode image BG. The operator inserts the biopsy needle 13 along the two-dimensional needle line N1_{2D}. The display image control unit 54 causes the two-dimensional tip display H_{2D} to be displayed on the B-mode image BG.

The distance calculation unit 52 calculates a distance D between the Doppler data and the tip portion of the biopsy needle 13. The distance calculation unit 52 calculates the distance D, based on the position of the Doppler data in the three-dimensional space and the position of the tip portion of the biopsy needle 13, both of which have been calculated by the position calculation unit 51.

The distance comparing unit 53 compares the distance D, the first threshold value Dth1 and the second threshold value Dth2. Here, as the distance D, distances between a plurality of points of Doppler data and the tip portion of the biopsy needle 13 may be calculated. When the distances D are calculated in plural form in this manner, the distance comparing unit 53 may compare a minimum distance Dmin of the calculated distances D and the first and second threshold values Dth1 and Dth2.

When D<Dth2 (Dth2<Dth1) (where D>Dth1), the display image control unit 54 causes the display unit 6 to display character information I about a warning including a character of “WARNING!” as shown in FIG. 5. The character information I about the warning is displayed beside a B-mode image BG. The character information I about the warning may have a color such as red or may blink.

Incidentally, the two-dimensional tip display H_{2D} shaped in square form is displayed in the B-mode image BG in FIG. 5.

When D<Dth1, the display image control unit 54 causes the display unit 6 to display a 3D image G_{3D} as shown in FIG. 6.

Incidentally, the display image control unit 54 may scale up or down the 3D image G_{3D} by the input of the operation unit 7 by the operator. The display image control unit 54 may rotate the 3D image G_{3D} by the input of the operation unit 7 by the operator. When the 3D image G_{3D} is rotated, the 3D image G_{3D} may automatically be kept continuous with the input of the operation unit 7 being taken as the timing. Alternatively, the 3D image G_{3D} may be rotated by an angle at which the operator desires to rotate, according to the input of the operation unit 7 by the operator.

According to the ultrasound diagnostic apparatus 1 of the present embodiment, when the distance D becomes smaller than the second threshold value Dth2, the character information I about the warning is displayed. Further, when the distance D becomes smaller than the first threshold value Dth1, the 3D image G_{3D} is displayed. It therefore becomes easy to insert the biopsy needle in such a manner as to avoid damage of vasa such as blood vessels.

Since the 3D image G_{3D} is displayed, it is possible to easily grasp a positional relationship between the biopsy needle 13 and the vas such as the blood vessel even if the biopsy needle 13 is not displayed in the B-mode image BG. On the other hand, since the 3D image G_{3D} is displayed only when the distance D becomes smaller than the first threshold value Dth1, the frame rate of the B-mode image BG can be maintained.

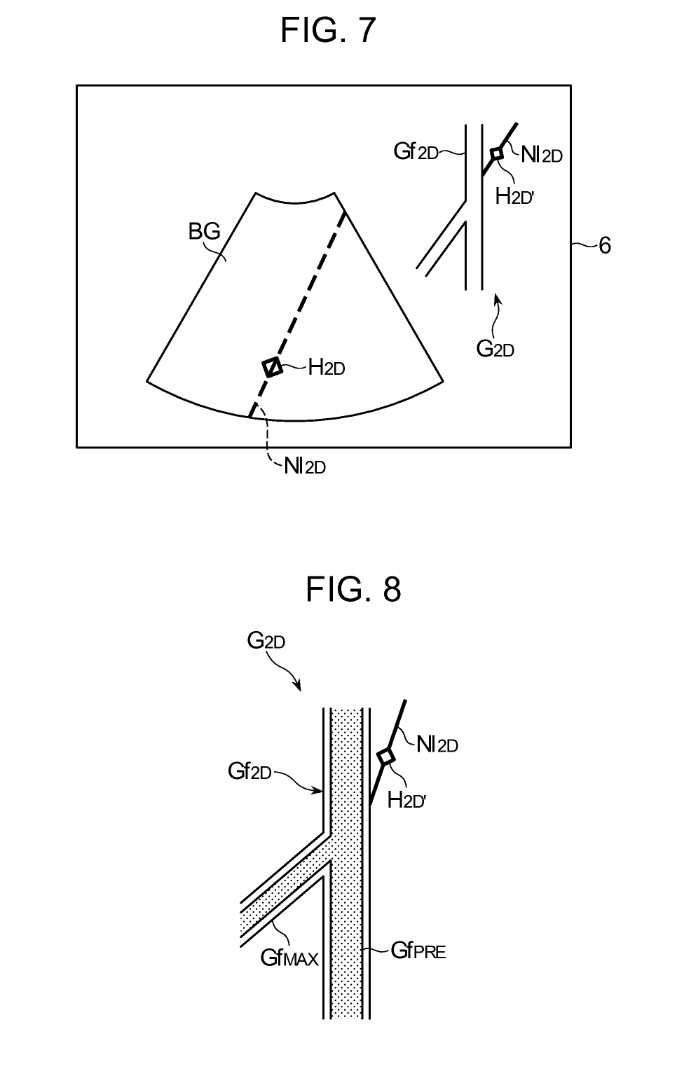

Modifications of the exemplary embodiment will next be explained. A first modification will first be described. The display image control unit 54 may cause a 2D image G_{2D} to be displayed as the image for the warning instead of the 3D image G_{3D} as shown in FIG. 7. The 2D image G_{2D} is includes a two-dimensional fluid image Gf_{2D}, a two-dimensional needle line N1_{2D} and a two-dimensional tip display H_{2D′}.

The display image control unit 54 generates two-dimensional fluid image data, based on the Doppler data and causes the two-dimensional fluid image Gf_{2D} to be displayed based on the two-dimensional fluid image data. The two-dimensional fluid image Gf_{2D} is, for example, an image relative to the same transmission/reception surface of ultrasound as the B-mode image BG.

A second modification will next be explained. The display image control unit 54 may generate three-dimensional fluid image data or two-dimensional fluid image data, based on data obtained by adding Doppler data corresponding to a plurality of frames at the same transmission/reception surface. In this case, an image of fluid maximum in diameter and an image of the present fluid may be displayed as the three-dimensional fluid image Gf_{3D} and the two-dimensional fluid image Gf_{2D}.

The 2D image G_{2D} will be illustrated by example. A two-dimensional fluid image Gf_{2D} comprised of an image Gf_{MAX} of fluid maximum in diameter and an image Gf_{PRE} of the present fluid may be displayed as shown in FIG. 8. For example, the image Gf_{MAX} is displayed in semitransparent form, and the image Gf_{PRE} is displayed in a predetermined color (the image Gf_{PRE} is represented in dots in FIG. 8).

Incidentally, although only the 2D image G_{2D} is shown in FIG. 8, the 2D image G_{2D} is displayed along with the B-mode image BG.

The image Gf_{MAX} is an image of fluid maximum in diameter in a cardiac cycle. This image Gf_{MAX} is updated every cardiac cycle. The image Gf_{MAX} may be generated based on the data obtained by adding the Doppler data in a period of the cardiac cycle. The image Gf_{MAX} may be generated based on data obtained by adding Doppler data in the systolic phase of the cardiac cycle.

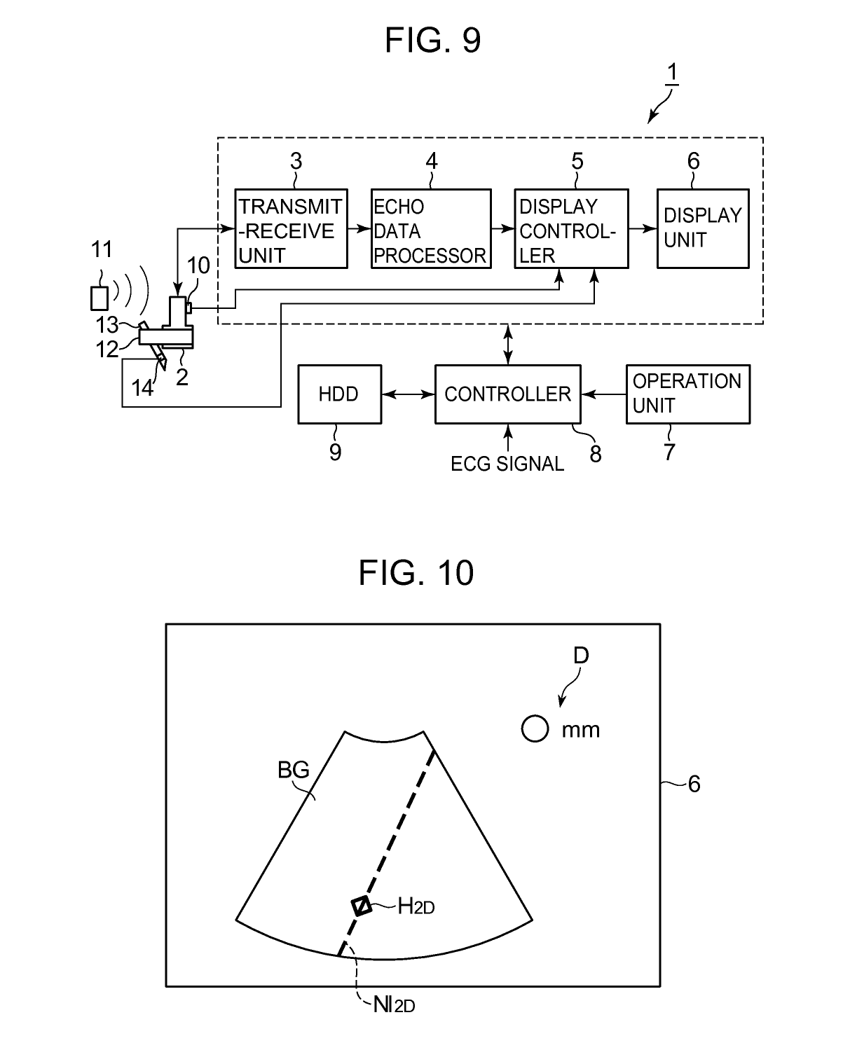

When the Doppler data in the systolic phase are added, an ECG (Electrocardiogram) signal is inputted to the controller 8 as shown in FIG. 9. The systolic phase is specified based on the ECC signal.

The image Gf_{PRE} is updated every frame. The diameter of fluid changes with a change in the flow rate of fluid.

A third modification will next be explained. The display image control unit 54 may cause the distance D to be displayed as shown in FIG. 10 as an image of warning displayed where the distance D has reached the second threshold value Dth2 or less, instead of the character information I about the warning and the 3D image G_{3D} or the 2D image G_{2D}.

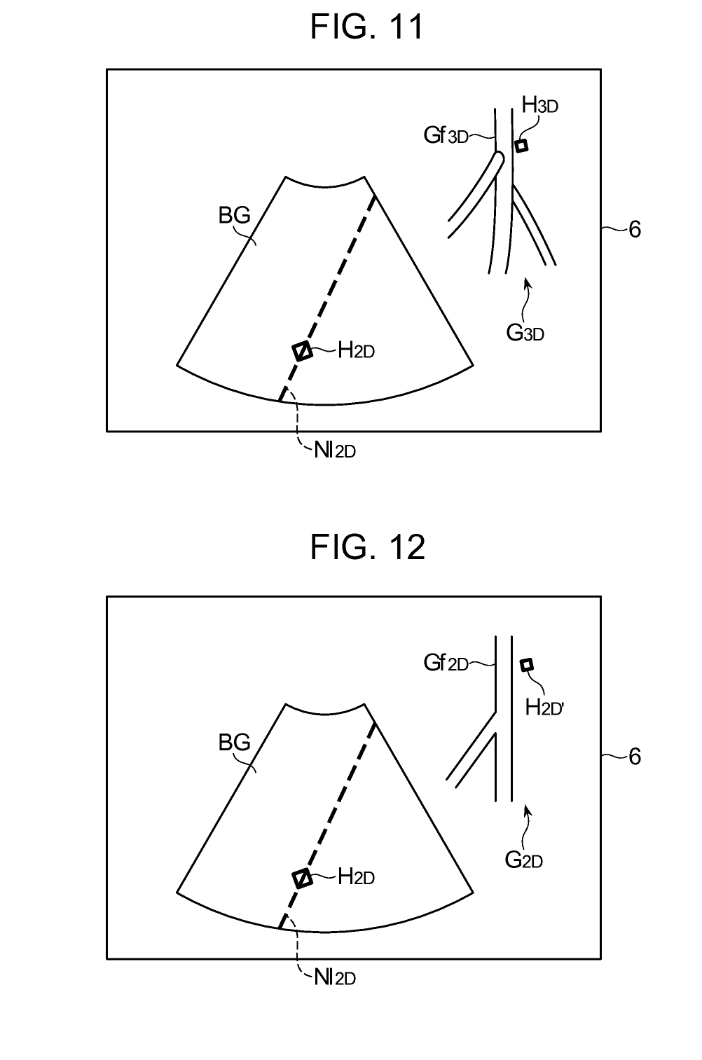

A fourth modification will next be explained. As shown in FIG. 11, the three-dimensional needle line N1_{3D} may not be displayed in the 3D image G_{3D}. As shown in FIG. 12, the two-dimensional needle line N1_{2D} may not be displayed in the 2D image G_{2D}.

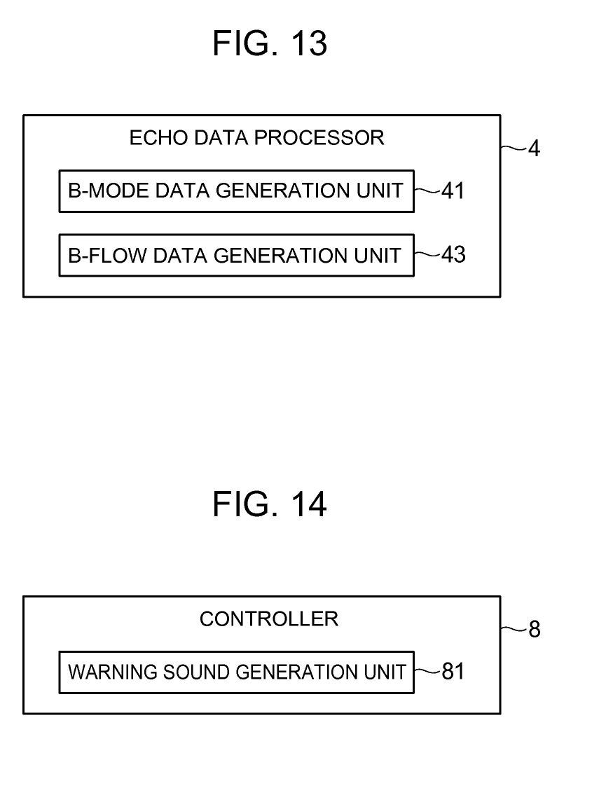

A fifth modification will next be described. As shown in FIG. 13, the echo data processor 4 may have a B-flow data generation unit 43 instead of the Doppler data generation unit 42. The B-flow data generation unit 43 performs B-flow processing on the echo data to generate B-flow data. In this case, the B-flow data is used instead of the Doppler data. The B-flow data is one example of an embodiment of fluid information data. The B-flow data generation unit 43 is one example of a fluid information data generation unit.

A sixth modification will next be explained. In the sixth modification, the controller 8 has a warning sound generation unit 81 as shown in FIG. 14. When the distance D becomes a predetermined threshold value or less, the warning sound generation unit 81 generates a warning sound instead of the display image control unit 54 causing an image of warning to be displayed.

Although exemplary embodiments are described above, it is needless to say that the invention may be modified in various forms within the scope and without changing the spirit of the invention. For example, switching to a mode in which the image of warning is not displayed may be performed by the input of the operation unit 7 by the operator.

Many widely different embodiments may be configured without departing from the spirit and the scope of the invention. It should be understood that the disclosure is not limited to the specific embodiments described in the specification, except as defined in the appended claims.

1. An ultrasound diagnostic apparatus comprising:
a fluid position detection unit configured to detect a position of fluid in a subject;
a biopsy needle position detection unit configured to detect a position of a biopsy needle to be inserted into the subject;
a distance calculation unit configured to calculate a distance between the fluid and the biopsy needle, based on the detected position of the fluid and the detected position of the biopsy needle; and
a notice unit configured to generate a warning when the distance calculated by the distance calculation unit is smaller than a predetermined threshold value.

2. The ultrasound diagnostic apparatus according to claim 1, wherein the fluid position detection unit comprises:
a first position sensor configured to detect a position of an ultrasound probe in a three-dimensional space with a predetermined point as an origin point;
a fluid information data generation unit configured to generate fluid information data about the subject, based on echo signals acquired by performing transmission/reception of ultrasound to and from the subject by the ultrasound probe; and
a position calculation unit configured to calculate a position of the fluid information data in the three-dimensional space, based on the detected position of the ultrasound probe.

3. The ultrasound diagnostic apparatus according to claim 2, wherein the fluid information data generation unit is configured to generate Doppler data, based on the echo signals.

4. The ultrasound diagnostic apparatus according to claim 2, wherein the fluid information data generation unit is configured to generate B-flow data, based on the echo signals.

5. The ultrasound diagnostic apparatus according to claim 1, wherein the biopsy needle position detection unit comprises:
a second position sensor configured to detect a position of the biopsy needle in a three-dimensional space with a predetermined point as an origin point; and
a position calculation unit configured to calculate the position of the biopsy needle in the three-dimensional space, based on a signal inputted from the second position sensor.

6. The ultrasound diagnostic apparatus according to claim 2, wherein the biopsy needle position detection unit comprises:
a second position sensor configured to detect a position of the biopsy needle in a three-dimensional space with a predetermined point as an origin point; and
a position calculation unit configured to calculate the position of the biopsy needle in the three-dimensional space, based on a signal inputted from the second position sensor.

7. The ultrasound diagnostic apparatus according to claim 1, including a distance comparing unit configured to compare the distance calculated by the distance calculation unit and the predetermined threshold value, wherein the notice unit is configured to generate the warning, based on a result of the comparison by the distance comparing unit.

8. The ultrasound diagnostic apparatus according to claim 7, wherein the predetermined threshold value includes a plurality of predetermined threshold values.

9. The ultrasound diagnostic apparatus according to claim 1, wherein the warning is character information displayed on a display unit.

10. The ultrasound diagnostic apparatus according to claim 1, wherein the warning is a warning image which is displayed on a display unit, the warning image including an image of the fluid and a display indicative of the position of the biopsy needle.

11. The ultrasound diagnostic apparatus according to claim 10, wherein the warning image is an image displayed by specifying a positional relationship between the fluid and the biopsy needle, based on the detected position of the fluid and the detected position of the biopsy needle.

12. The ultrasound diagnostic apparatus according to claim 11, wherein the warning image is a three-dimensional image.

13. The ultrasound diagnostic apparatus according to claim 11, wherein the warning image is a two-dimensional image.

14. The ultrasound diagnostic apparatus according to claim 10, wherein an image of fluid maximum in diameter is displayed as the image of the fluid.

15. The ultrasound diagnostic apparatus according to claim 14, wherein the image of the fluid maximum in diameter is generated based on data obtained by adding fluid information data generated based on echo signals obtained by transmitting ultrasound to the subject over a plurality of frames with respect to the same transmission/reception surface of ultrasound.

16. The ultrasound diagnostic apparatus according to claim 15, wherein the image of the fluid maximum in diameter is generated based on data obtained by adding the fluid information data in a systolic phase of a cardiac cycle over a plurality of frames.

17. The ultrasound diagnostic apparatus according to claim 9, wherein a real-time ultrasound image is displayed on the display unit.

18. The ultrasound diagnostic apparatus according to claim 1, wherein the warning is a display of the distance calculated by the distance calculation unit.

19. The ultrasound diagnostic apparatus according to claim 1, wherein the warning is sound.

20. A method for an ultrasound diagnostic apparatus, the method comprising:
detecting a position of fluid in a subject;
detecting a position of a biopsy needle to be inserted into the subject;
calculating a distance between the fluid and the biopsy needle, based on the detected position of the fluid and the detected position of the biopsy needle.
generating a warning when the distance calculated is smaller than a predetermined threshold value.
